(19) United States
(12) Patent Application Publication
(10) Pub. No.: US 2024/0104724 A1
(43) Pub. Date: Mar. 28, 2024

(54) RADIOMICS STANDARDIZATION

(71) Applicant: THE JOHNS HOPKINS UNIVERSITY, Baltimore, MD (US)

(72) Inventors: Jianan GANG, Baltimore, MD (US); Joseph Webster STAYMAN, Baltimore, MD (US)

(73) Assignee: THE JOHNS HOPKINS UNIVERSITY, Baltimore, MD (US)

(21) Appl. No.: 18/255,630

(22) PCT Filed: Dec. 3, 2021
(86) PCT No.: PCT/US2021/061729

§ 371 Date: Jun. 2, 2023

Related U.S. Application Data
(60) Provisional application No. 63/121,646, filed on Dec. 4, 2020.

(51) Int. Cl. G06T 7/00; G16H 30/40

(52) U.S. Cl. CPC G06T 7/0012; G16H 30/40; A61B 6/583

(57) ABSTRACT

Techniques for radiomics standardization for patient scan data obtained by a particular imaging device are presented. The techniques include acquiring, using the particular imaging machine, the patient scan data; obtaining unstandardized radiomics for the patient scan data; recovering standardized radiomics for the patient scan data based on at least: the patient scan data, the unstandardized radiomics for the patient scan data, and calibration phantom data for the particular machine obtained using at least one calibration phantom; and outputting the standardized radiomics.

### RELATED APPLICATION

This application claims the benefit of, and priority to, U.S. Provisional Patent Application No. 63/121,646, entitled, “Radiomics Standardization,” and filed Dec. 4, 2021, which is hereby incorporated by reference in its entirety.

### STATEMENT OF GOVERNMENT SUPPORT

This invention was made with government support under grant CA219608 awarded by the National Institutes of Health (NIH). The government has certain rights in this invention.

### FIELD

This disclosure relates generally to medical imaging radiomics.

### BACKGROUND

Medical imaging includes Computed Tomography (CT), Magnetic Resonance Imaging (MRI), x-rays, ultrasound, microscopy, and other techniques. Medical imaging may produce two-dimensional images or three-dimensional volumes constructed from multiple two-dimensional images. Such three-dimensional volumes may be sliced in any of a variety of ways to obtain two-dimensional images.

Radiomics, or medical imaging biomarkers, is an active area of research and development. Radiomics models have been applied in a wide range of diagnostics, classification tasks, and disease scoring. Radiomics is advantageous for efficient radiology workflow, for example, reducing errors. Further, radiomics can highlight important features and provide additional information in challenging diagnostic cases. Radiomics has been extensively investigated in oncology diagnostics and is finding applications in other diseases in a wide range of organ systems. Radiomics can significantly boost the utility of imaging studies by using quantitative image features to infer underlying tumor biology and predict patient outcomes. For lung imaging in particular, radiomics has demonstrated utility in cancer diagnosis, prognosis, and precision medicine, as well as in the evaluation of a wide range of lung diseases including COPD, asthma, and tuberculosis.

### SUMMARY

According to various embodiments, a method of radiomics standardization for patient scan data obtained by a particular imaging device is presented. The method includes acquiring, using the particular imaging machine, the patient scan data; obtaining unstandardized radiomics for the patient scan data; recovering standardized radiomics for the patient scan data based on at least: the patient scan data, the unstandardized radiomics for the patient scan data, and calibration phantom data for the particular machine obtained using at least one calibration phantom; and outputting the standardized radiomics.

Various optional features of the above embodiments include the following. The particular imaging machine can include at least one: x-ray machine, computed tomography machine, magnetic resonance imaging machine, or ultrasound machine. The patient scan data can include a two-dimensional slice of a three-dimensional volume constructed from raw patient scan data. The patient scan data can include raw patient scan data. The method can include: providing the patient scan data and the calibration phantom data to a trained image property predictor; and obtaining noise and resolution characteristics for the particular machine from the trained image property predictor; wherein the recovering the standardized radiomics comprises recovering the standardized radiomics based on the patient scan data, the unstandardized radiomics for the patient scan data, and the noise and resolution characteristics. The recovering the standardized radiomics can include: providing the patient scan data, the unstandardized radiomics for the patient scan data, and the calibration phantom data for the particular machine to a machine learning model trained using a training corpus comprising radiomics in association with example scan data and calibration phantom data, whereby the machine learning model provides the standardized radiomics. The recovering can include: deblurring an image corresponding to the patient scan data to produce a deblurred image; determining radiomics for the deblurred image; determining radiomics for noise of the deblurred image; and deconvolving the radiomics for the deblurred image with the radiomics for the noise of the deblurred image. The recovering can include: passing an image corresponding to the patient scan data to a first machine learning model trained to deblur images to obtain a deblurred image; computing radiomics for the deblurred image; passing the radiomics for the deblurred image to a second machine learning model trained to remove noise, whereby the standardized radiomics are obtained. The radiomics can include standardized radiomics comprise a grey-level co-occurrence matrix. The outputting can include causing the standardized radiomics to be input to a radiomics model for clinical decision making.

According to various embodiments, a system for radiomics standardization for patient scan data obtained by a particular imaging device is presented. The system includes at least one electronic processor that executes instructions to perform operations comprising: acquiring the patient scan data produced by the particular imaging machine; obtaining unstandardized radiomics for the patient scan data; recovering standardized radiomics for the patient scan data based on at least: the patient scan data, the unstandardized radiomics for the patient scan data, and calibration phantom data for the particular machine obtained using at least one calibration phantom; and outputting the standardized radiomics.

Various optional features of the above embodiments include the following. The particular imaging machine can include at least one: x-ray machine, computed tomography machine, magnetic resonance imaging machine, or ultrasound machine. The patient scan data can include a two-dimensional slice of a three-dimensional volume constructed from raw patient scan data. The patient scan data can include raw patient scan data. The operations can further include: providing the patient scan data and the calibration phantom data to a trained image property predictor; and obtaining noise and resolution characteristics for the particular machine from the trained image property predictor; wherein the recovering the standardized radiomics comprises recovering the standardized radiomics based on the patient scan data, the unstandardized radiomics for the patient scan data, and the noise and resolution characteristics. The recovering the standardized radiomics can include: providing the patient scan data, the unstandardized radiomics for the patient scan data, and the calibration phantom data for the particular machine to a machine learning model trained using a training corpus comprising radiomics in association with example scan data and calibration phantom data, whereby the machine learning model provides the standardized radiomics. The recovering can include: deblurring an image corresponding to the patient scan data to produce a deblurred image; determining radiomics for the deblurred image; determining radiomics for noise of the deblurred image; and deconvolving the radiomics for the deblurred image with the radiomics for the noise of the deblurred image. The recovering can include: passing an image corresponding to the patient scan data to a first machine learning model trained to deblur images to obtain a deblurred image; computing radiomics for the deblurred image; passing the radiomics for the deblurred image to a second machine learning model trained to remove noise, whereby the standardized radiomics are obtained. The radiomics can include standardized radiomics comprise a grey-level co-occurrence matrix. The outputting can include causing the standardized radiomics to be to be input to a radiomics model for clinical decision making.

### BRIEF DESCRIPTION OF THE DRAWINGS

Various features of the embodiments can be more fully appreciated, as the same become better understood with reference to the following detailed description of the embodiments when considered in connection with the accompanying figures, in which:

FIG. 1 is a schematic diagram of a radiomics imaging chain according to various embodiments;

FIG. 2 depicts schematic diagrams of a radiomics prediction model 202 and of a radiomics recovery model 204 for linear imaging systems according to various embodiments;

FIG. 3 is a schematic diagram of a grey-level co-occurrence matrix radiomics prediction model and recovery model according to various embodiments;

FIG. 4 depicts schematic diagrams of a radiomics prediction model 402 and of a radiomics recovery model for imaging systems with possible data-dependent noise and/or resolution according to various embodiments;

FIG. 5 depicts an anthropomorphic calibration phantom according to various embodiments;

FIG. 6 depicts schematic diagrams of a radiomics prediction model 602 and of a radiomics recovery model 604 for highly non-linear imaging systems according to various embodiments;

FIG. 7 depicts example calibration phantoms with attenuation profiles and textures emulating lung tissues according to various embodiments;

FIG. 8 is a flow diagram for a method of radiomics standardization according to various embodiments; and

FIG. 9 is a schematic diagram for a system for implementing a method of radiomics standardization according to various embodiments.

### DESCRIPTION OF THE EMBODIMENTS

Reference will now be made in detail to example implementations, illustrated in the accompanying drawings. Wherever possible, the same reference numbers will be used throughout the drawings to refer to the same or like parts. In the following description, reference is made to the accompanying drawings that form a part thereof, and in which is shown by way of illustration specific exemplary embodiments in which the invention may be practiced. These embodiments are described in sufficient detail to enable those skilled in the art to practice the invention and it is to be understood that other embodiments may be utilized and that changes may be made without departing from the scope of the invention. The following description is, therefore, merely exemplary.

Despite the potential of radiomics, it is widely acknowledged that a major challenge to clinical usage is the robustness and repeatability of radiomics models. Such concerns arise from variability in radiomics values from each step in the imaging chain including: (1) data collection from different imaging systems and protocols, (2) lack of standardization in image formation and processing, and (3) lack of standardization in radiomics computation and reporting of such models. The latter two can be resolved through a concerted effort in the research community and have in fact motivated several initiatives and guidelines to standardize definitions, methodologies, and reporting. The first, however, reflects a fundamental technical challenge, as radiomics values are inherently affected by the quality of the image data, which is in turn affected by acquisition techniques, reconstruction parameters, and scanner specifications.

Radiomics relies on medical image data which not only contain variability due to noise, but also differing biases (e.g. including different spatial resolutions) induced by the use of hardware from different vendors, different acquisition protocols, and different data processing as part of image formation or post-processing. The problem of image-chain-based variability in radiomics features is common among all imaging modalities with a large variety of scanners, acquisitions protocols, and post-processing. Complex noise and bias characteristics have become particularly exaggerated with the increased use of sophisticated data processing schemes, e.g., sparse acquisitions and compressed sensing in MRI model-based iterative methods in computer tomography and nuclear imaging, and machine learning methods in all modalities. Variability in image data is well-known in x-ray computed tomography. Even with conventional linear processing (e.g., filtered backprojection), noise properties of the image are patient and protocol dependent, and non-stationary (e.g., varying contrast with kVp, increased noise with larger patients, etc.). With model-based iterative reconstruction and machine learning methods, image properties can also have significant dependencies in spatial resolution, often characterized as contrast-dependent and space-variant.

One school of thought is that the radiomics model itself can handle variability, that is, given sufficient training data of different varieties, the model can learn to handle the difference in image quality and radiomics features. However, in a real-world clinical setting, there are numerous scanner-specific, institution-specific, or even radiologist-specific imaging and reconstruction protocols. Curating sufficient training data for each case and retraining the model to handle such variability is not feasible.

Several studies have evaluated the effect of mixing imaging conditions on radiomics model performance with limited success, e.g., using smooth and sharp filtered backprojection kernels. The performance of the radiomics models were found to decrease significantly and the authors have suggested against mixing imaging conditions. Numerous studies have emerged to call attention to the dependence of radiomics feature variability on imaging conditions and highlighting such variability as a major challenge to radiomics research.

Attempts to solve these issues tend to fall into one of three categories: (1) harmonizing acquisition and processing protocols, (2) identification of radiomics that are robust across different protocols and imaging conditions, and (3) normalization of radiomics to a standard imaging protocol. With a diversity of vendors and imaging hardware, the first has not been achieved, and the second limits the number of available features. Many normalization strategies (the third category) have been proposed but, thus far, it is unclear whether they can account for the full range of imaging systems and protocol diversity.

Some embodiments provide a solution by treating radiomics computation as an additional step in the imaging chain. Some embodiments utilize an end-to-end prediction framework that relates how each imaging parameter affects radiomics values. Some embodiments can not only predict radiomics from arbitrary imaging conditions, but also invert the model and normalize values to a standard protocol, thereby achieving robust and repeatable radiomics.

These and other features and advantages are shown and described in reference to the figures as set forth presently.

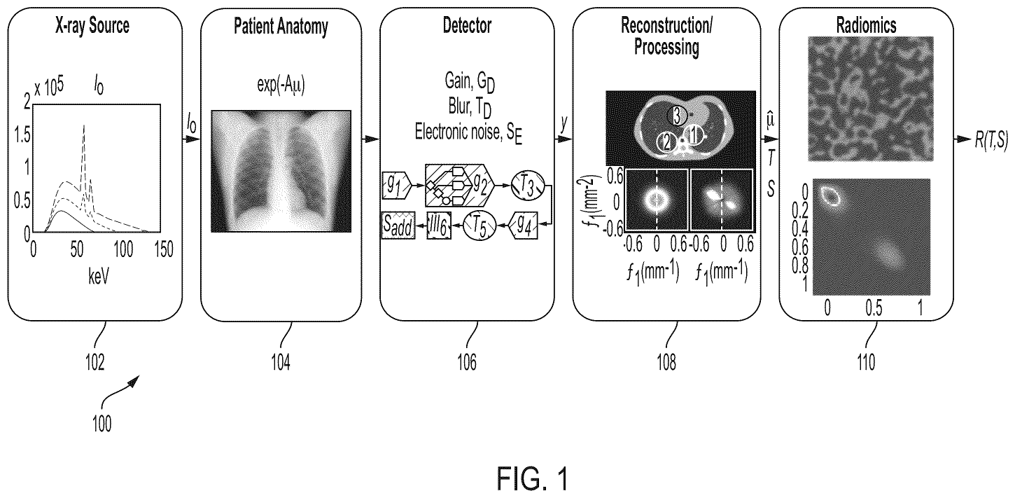

FIG. 1 is a schematic diagram of a radiomics imaging chain 100 according to various embodiments. In general, performance of imaging system properties may be described in terms of noise and bias (where spatial resolution metrics often describe non-DC biases and the limits on high spatial frequencies). Such imaging properties may be considered and quantified using theoretical end-to-end image quality models that describe signal and noise propagation through the physical and mathematical processes of image formation. As shown in FIG. 1, such models may include an x-ray source stage 102, a patient anatomy stage 104, a detector stage 106, and a reconstruction/processing stage 108. Such models are powerful tools that relate changes in the imaging conditions (e.g., imaging technique, scanner specification, reconstruction parameters, etc.) to changes in the output image properties. Because radiomics are directly computed from image data, their values are necessarily dependent on the underlying image properties. Thus, some embodiments consider radiomics using a direct extension of such end-to-end models (e.g., including a fifth stage, such as block 110 in FIG. 1). Such models may include image property propagation (e.g., noise S and resolution 7) through different stages of the imaging chain, as depicted in FIG. 1. That is, such models may quantify noise S and resolution T through each stage.

Usage of such end-to-end models permits embodiments to not only predict radiomics variability as a function of imaging condition, but also to standardize radiomics values to a common baseline via inversion of the models. Thus, predictive models are innovative in that they have direct application in the optimization and design of imaging protocols best suited for estimation of specific radiomics. For example, what is good for a radiomics model in terms of performance may be mismatched with what is good for general radiologists' performance. Inverted (that is, recovery) models provide a concrete and mathematically rigorous way to estimate the underlying tissue radiomics, thereby providing a common foundation across data from different sources (vendor, protocol, etc.). Thus, some embodiments utilize a novel paradigm for standardization in which the underlying radiomics themselves are estimated.

There are some distinct advantages in defining standardization as a radiomics estimation problem, as opposed to image standardization, including: (1) The parameter space for radiomics estimation is much smaller than the joint image denoising/blur deconvolution problem. This generally represents a better conditioned inversion. (2) Image correction methods are focusing on providing the underlying true image. Radiomics are only trying to capture specific features (e.g., textures of a certain scale and/or directionality). Thus, some embodiments do not include solving the more difficult problem of estimating the true image (e.g., deblurring/denoising in all directions) and instead focus on the problem of the radiomics themselves. (3) Treating the problem as a radiomics estimation problem permits modeling of the prior distribution of radiomics, e.g., known sparsity in a gray-level cooccurrence matrix, etc. Lastly, focusing on end-to-end radiomics estimation formalizes the quantitation problem. If there are sub-resolution “signatures” of specific disease processes (which cannot be seen visually), modeling and estimation of the underlying radiomics may demonstrate concretely where those signatures arise.

Embodiments may utilize models that are modular and general, and that can encompass combinations of hardware specifications, as well as both linear and nonlinear reconstruction and processing algorithms. The resolution and noise can either be derived from a fully analytic model based on known system parameters, or obtained from empirical measurement of one or more calibration phantoms. Moreover, the prediction framework may be inverted to use measured radiomics in the blurred and noisy data, R(T,S), to obtain the ground truth radiomics values or radiomics values of a standardized imaging protocol, R(T₀,S₀). Thus, some embodiments include explicit estimation of the underlying radiomics through rigorous modeling of system dependencies that lead to variability, e.g., in noise and resolution. Modeling such dependencies allows for aggregation from disparate data sources with improved radiomics quantitation.

Imaging systems may be linear, weakly nonlinear, or highly nonlinear.

Examples of linear systems include standard filtered backprojection, with noise and resolution characterized by noise-power spectrum and modulation transfer function. Such systems can be considered linear and shift-invariant. Some radiomics computations are also linear (e.g., those based on linear decompositions, such as Fourier, Hadamard, or wavelet transforms, etc.). Computer tomography systems employing locally linearizable model-based reconstructions (like quadratically penalized-likelihood) may also be considered linear. Certain classes of radiomics (e.g., GLCM and histogram-based methods) can also be described by linear operations on either the underlying images or on the radiomics themselves.

Examples of weakly nonlinear systems include model-based iterative reconstruction, which can be modeled as locally linear.

Examples of highly nonlinear system include those that utilize deep learning, e.g., for denoising.

In the following, embodiments are disclosed for use with each of these types of imaging systems. Note that the selection of the appropriate embodiment for a given situation may depend on prior knowledge of the imaging system and whether radiomics computations can be analytically modeled.

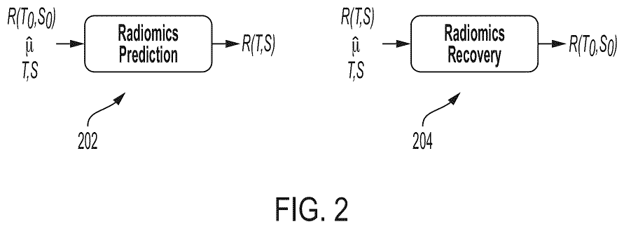

FIG. 2 depicts schematic diagrams of a radiomics prediction model 202 and of a radiomics recovery model 204 for linear imaging systems, according to various embodiments. In FIG. 2, \hat{μ} represents patient scan data (which may be raw scan data or a volume or image reconstruction), R represents radiomics, T represents resolution of a particular imaging system, S represents noise of a particular imaging system, T₀ represents resolution of a standardized (idealized) imaging system, and S₀ represents noise of a standardized (idealized) imaging system.

Models 202 and 204 are applicable where the imaging system is at least locally linear and noise is stationary. System blur may be shift invariant or involve simple shift variance as a result of focal spot blur. The image noise S and resolution T can be obtained from existing linear systems models given knowledge of the system parameters, or from phantom measurements of noise and resolution. The inputs for the prediction model 202 and the recovery model 204 may therefore include known system blur T (shift variant or invariant) and noise S (magnitude and correlation), as well as the (local) patient scan (\hat{μ}) from which radiomics features (R) are calculated. According to some embodiments, raw scan data y may be substituted for the patient scan \hat{μ}.

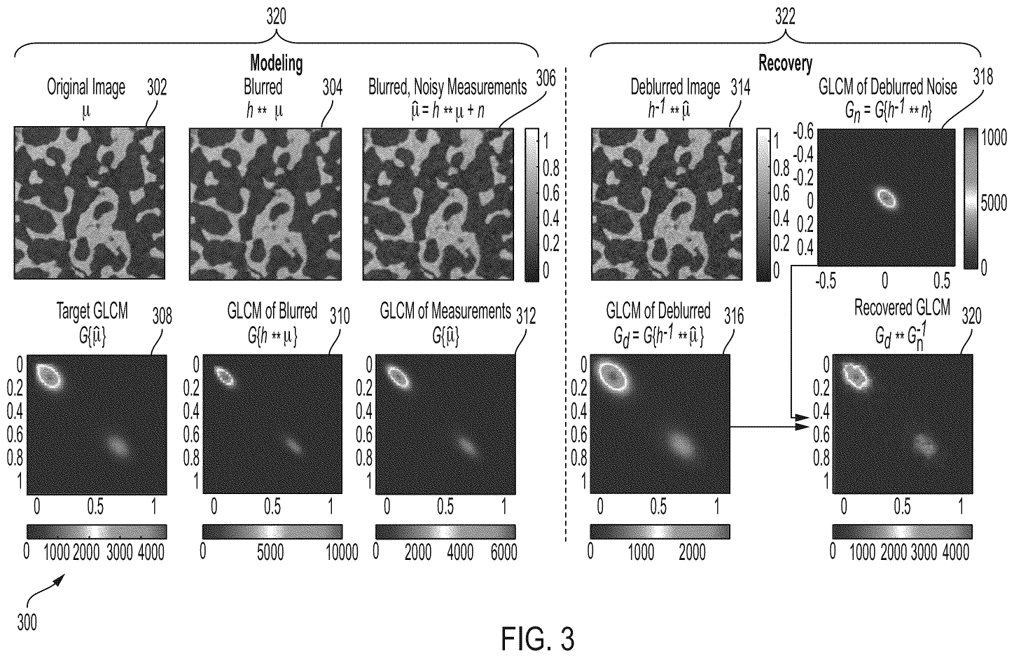

For example, Gray-Level Co-Occurrence Matrix (GLCM) is an example category of radiomics metrics that can be modeled analytically. For linear imaging systems where the resolution and noise is known, the radiomics recovery procedure may be implemented as described below in reference to FIG. 3.

FIG. 3 is a schematic diagram 300 of a GLCM radiomics prediction model 320 and recovery model 322 according to various embodiments. Such embodiments may utilize a general prediction model 320 that applies to arbitrary locally shift-invariant/stationary imaging systems and conditions. The prediction model 320 may be provided with the point spread function of the system (h) and noise (n) for the particular imaging conditions. Such information can be obtained using empirical measurements of spatial resolution and noise, or derived from established system models (e.g., cascaded system analysis). To model noisy, blurry data, the prediction model 320 may begin with a ground truth image \hat{μ} 302 and apply the known blur to obtain blurred image 304. For additive Gaussian noise for all voxels, i.e., n˜N(0,K) with covariance K, realizations of that noise may be generated (i.e., convolving with an appropriate kernel to impart the given covariance) and added to the blurred image to form noisy measurements 306. Note that, as shown in FIG. 3, blur narrows the original GLCM 308 toward the diagonal as seen in image 310, while additive noise tends to spread values. Importantly, both blur and noise prevent the GLCM 312 determined directly from measurements from being accurate.

The GLCM may be recovered from an original image using recovery model 322 as follows. The effect of blur on the GLCM is potentially complex and depends on the underlying image (\hat{μ}). One can attempt to recover the original image using a deblurring operation to yield deblurred image 314. Note that this process is noise magnifying and results in the following form: h⁻¹**\hat{μ}=μ+h⁻¹**n. Note further that deblurring increases noise in the deblurred image and broadens the GLCM 316 of the deblurred image 314. However, additive Gaussian noise has the effect of convolving the original GLCM with the GLCM of that additive noise. Thus, one can model the noise in the deblurred image 314, form the GLCM 318 of the noise of the deblurred image, and deblur the GLCM directly by deconvolving the GLCM 316 of the deblurred image 314 with the GLCM 318 of the noise of the deblurred image to recover the target GLCM 320. Such deconvolutions may be implemented in a variety of ways including classical Fourier methods, iterative model-based deconvolution, or neural network based techniques. This process is illustrated in FIG. 3 using simulated data with exactly known blur and noise distributions of the original measurements.

While noisy and blurry measurements yield different GLCM results than the original target data, embodiments can largely recover the GLCM. This processing is noise magnifying; however, the approach as shown and described in reference to FIG. 3 uses only very simple regularization and constraints to deconvolve the noise GLCM. This approach can standardize GLCM-based radiomics across an image database if models/measurements of both the noise and resolution are available. Such standardization would have dramatic impact in reducing variability in a broad range of computer-aided diagnosis and radiomics data science.

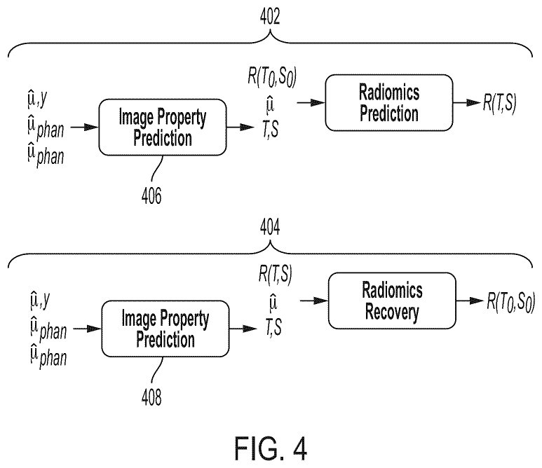

FIG. 4 depicts schematic diagrams of a radiomics prediction model 402 and of a radiomics recovery model 404 for imaging systems with possible data-dependent noise and/or resolution according to various embodiments. In general, many processing strategies introduce additional object dependencies. These can manifest image-dependent shift-variant/non-stationary image properties, and include model-based iterative reconstruction methods like penalized-likelihood reconstruction with quadratic penalty (PLQ) that are locally linear but have contrast-dependencies over a large regions of interest (e.g., radiomics models covering an entire lung). The interaction of statistical weighting and regularization in PLQ yields shift-variant resolution properties that are dependent on the noise in projections passing through each point in the image volume. Prediction and recovery under this imaging scenario relies on accurate characterization of the space-variant image properties. Using image quality models that incorporate the aforementioned effects, some embodiments can spatially sample the image volume to obtain pointwise, local predictions of noise and resolution. Inversion and recovery methods are then performed as depicted in FIG. 4, e.g., as a generalization of the techniques as shown and described above in reference to FIG. 3.

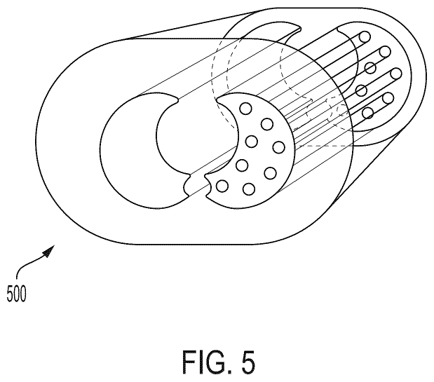

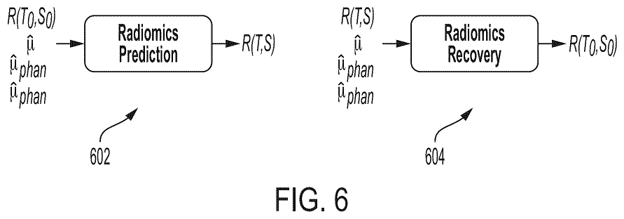

For weakly non-linear imaging systems, e.g., with possible noise and/or resolution data dependence and/or that can be modeled as locally linear as considered in reference to FIG. 4, the resolution and noise characteristics may not be obtained (or obtainable) directly from standard noise resolution measurements. Instead, such embodiments may utilize a dedicated radiomics calibration phantom with known ground truth, μₚₕₐₙ, and use the phantom scan (μₚₕₐₙ) to obtain the system blur and noise characteristics via image property predictors 406, 408. Example such phantoms are shown and described below in reference to FIGS. 5 and 6. The patient scan (\hat{μ}) (or the raw measurement data (y)) are also inputs to the image property predictors 406, 408 in order to account for potential data dependence (e.g., through local statistical weights) of image properties. The image property predictors 406, 408 may be implemented as convolutional neural networks according to some embodiments. Such a neural network may be trained with sets of phantom ground truth μₚₕₐₙ, phantom scan \hat{μ}ₚₕₐₙ, and patient scan \hat{μ} (or the raw patient measurement data (y)) paired with associated image noise S and resolution T characteristics. These calibrated image properties, e.g., T and S, are then passed into the respective prediction/recovery portions of models 402 and 404 together with the radiomics features and the (local) patient image used for radiomics calculation.

This scenario permits application of the radiomics prediction and recovery framework in situations where closed-form inputs for T, S may not be directly specified. For example, this may arise in imaging systems with proprietary (“black box”) processing, or in scenarios with more complex noise and resolution properties including contrast dependence of the point spread function, etc.

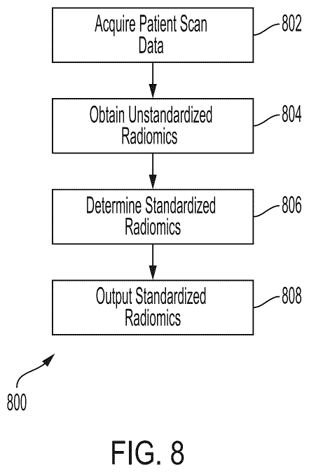

FIG. 5 depicts an anthropomorphic calibration phantom 500 according to various embodiments. Phantom 500 may be used as shown and described in reference to FIGS. 6 and 8, and particularly FIG. 4, for example. To model the spatially varying noise and resolution properties of nonlinear systems, particularly weakly nonlinear systems, some embodiments utilize a spatial sampling technique where noise (flat regions) and resolution (points/lines) are placed throughout the image field-of-view. Because of the inherent dependence on patient size of these methods, some embodiments use a digital calibration phantom such as phantom. Phantom 500 represents an example such phantom for use in calibrating systems used for lung scans has attenuation characteristics that are representative of those of lung computer tomography patients of different sizes. Phantom 500 as shown in FIG. 5 includes a sampling of stimuli positions for noise-power spectrum (NPS) and modulation transfer function (MTF) targets. Multiple such phantoms may be used, with varying representative thorax sizes.

FIG. 6 depicts schematic diagrams of a radiomics prediction model 602 and of a radiomics recovery model 604 for highly non-linear imaging systems according to various embodiments. In general, highly nonlinear image reconstruction/processing (e.g., model-based iterative reconstruction with edge-preserving regularizers, deep learning denoising) can yield highly image-dependent image properties. Classic metrics of noise and resolution are often no longer applicable. Thus, models 602 and 604 represent highly nonlinear imaging systems where the resolution and noise are not easily calibrated or described. This may include systems with deep learning reconstructions/processing or highly nonlinear regularization/noise control strategies.

Models 602 and 604 directly provide the calibration phantom ground truth, phantom scan, and the patient scan into the prediction/recovery framework. Thus, prediction model 602 and recovery model 604 combine all of these inputs and process them appropriately to model/return the radiomics to a particular image quality level. This may be achieved using a general neural network model for both prediction and recovery. Such a network is trained with radiomics for both truth/standardized image quality inputs as well as calibration (texture) phantoms with known baseline and scans on particular systems, protocols, and other system characteristics.

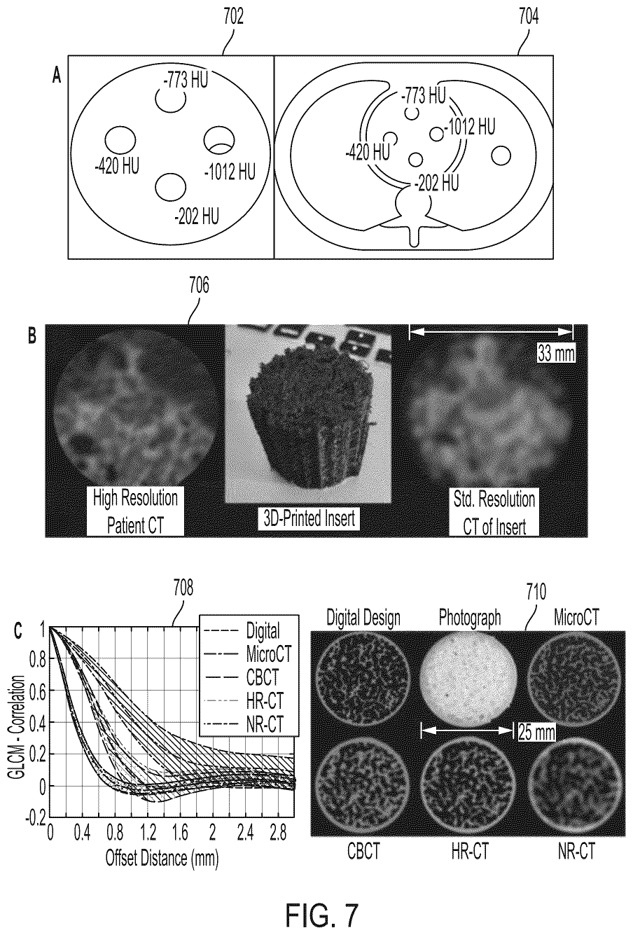

Some embodiments characterize the performance of highly nonlinear systems using perturbation response/specific stimuli of interest rather than traditional metrics of resolution. To probe these effects, some embodiments insert texture stimuli of a calibration phantom in order to characterize the system response to relevant textures. These stimuli reveal both signal transfer in the conventional sense, and radiomics transfer (of the underlying biological “signal”). In particular, some embodiments use high resolution texture stimuli that are representative of different lung patterns (e.g., normal, fibrotic, ground glass, honeycomb) but that are distinct from the set of features used for validation. Examples of such texture stimuli are shown and described below in reference to FIG. 7.

Using pairs of “known” input textures and “corrupted” output textures permits the development of a learned transfer model (e.g., image property predictors 406, 408) that captures the more complex image properties and the particular effects on each stimulus. The trained transfer models can then be integrated within a greater recovery model that seeks not an inversion of the similarly corrupted computer tomography data to “clean/true” computer tomography data, but instead seeks to recover the underlying radiomics (a potentially much easier inversion). There are several ways to integrate the transfer model including: (1) explicit integration as an untrained layer within the recovery network, (2) providing the input-output pairs themselves as auxiliary inputs—requiring the recovery network to learn both the transfer model and the radiomics inversion, and (3) integration of not the forward transfer model, but instead the inverse transfer model, again, as an untrained layer in a larger recovery network. The third option effectively embeds image inversion (“corrupt” “clean”) within a larger network that can handle the inversion difficulties as they relate to providing accurate radiomics.

FIG. 7 depicts example calibration phantoms 702, 706, 710 with attenuation profiles and textures emulating lung tissues according to various embodiments. The calibration phantoms of FIG. 7 may be used as shown and described in reference to FIGS. 4, 6, and particularly FIG. 8. The calibration phantoms of FIG. 7 may be implemented as, by way of non-limiting example, 3D printed phantoms with attenuation profiles and textures appropriate for emulation of lung tissues and reproduction of specific radiomics metrics.

As shown, FIG. 7 depicts Fused Deposition Modelling (FDM) phantom 702 and corresponding computer tomography scan 704 with lung attenuation values. That is, phantom 702 is designed to mimic lung attenuation values. FIG. 7 also depicts three-dimensional printed lung textures 706 based on high-resolution patient scans. That is, phantom 706 is based on patient data. FIG. 7 further depicts textured inserts 710 produced using stereolithography (SLA) printing where the radiomics metric (GLCM-correlation) of the digital design and three-dimensional print agree over a range of offset values. Graph 708 depicts that different computer tomography systems (CBCT, high-resolution, and normal-resolution computer tomography) variably reproduce such radiomics.

A range of structures and stimuli may be present in a physical phantom. For flexibility throughout the development effort, some embodiments utilize a modular phantom design, where specific target stimuli inserts may be placed throughout a larger anthropomorphic phantom. Such a modular design allows for: (1) Establishment of ground truth for the inserts via a computer tomography scan, (2) Refinement and/or alteration of phantoms (e.g., with new inserts), and (3) Re-use of the larger anthropomorphic bulk of the phantom, e.g., between calibration and validation.

A number of phantom construction techniques may be used, e.g., traditional manufacturing methods and additive manufacturing techniques. A summary of such techniques appears in the below Table.

TABLE

| Fabrication | | | |
|---|---|---|---|
| Method | Targets | Feature Sizes | Contrast |
| Casting and | Lines, wires, small beads | Down to ~100 μm | High (metal/plastic) |
| machining | | | |
| | Edges, line pairs | Down to ~300 μm | High and low |
| | Basic textures (packed | Down to ~1.5 mm | High and low |
| | spheres) | | |
| | Available textures | Variable, uncontrolled | Variable |
| | (sponges, etc.) | | |
| 3D printing - | Prodedural textures & | Down to ~150 μm | High and low |
| SLA | based on patient scans | | (plastic/solution) |
| 3D printing - | Prodedural textures & | Down to ~250 μm | Variable based on fill |
| FDM | based on patient scans | | percentage |
| 3D printing - | Prodedural textures & | Down to ~100 μm | Low |
| Polyjet | based on patient scans | | |

3D-printed phantoms may be subject to unknowns in the printing process that require characterization. Thus, according to some embodiments, fabricated texture inserts may be scanned using a high-resolution microCT to establish ground truth. To accommodate contrast differences due to varying technique (e.g., kVp) between microCT and the computer tomography targets, uniform samples may be also scanned and linear fitting may be applied to find ground truth at the computer tomography technique. Print-to-print variability of targets may be quantified by measuring the variability (e.g., standard deviation) of radiomics of the microCT to determine if ground truth values need to be print-specific (e.g., for multiple copies of an insert in a phantom, or in different copies of the same stimulus across collaborating sites).

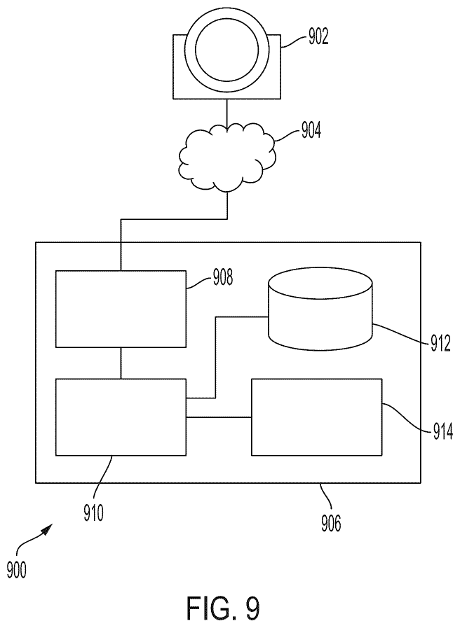

FIG. 8 is a flow diagram for a method 800 of radiomics standardization for a particular imaging machine according to various embodiments. Method 800 may be practiced using the system shown and described below in reference to FIG. 9. Method 800 may implement one or more of radiomics recovery model 204, 404, or 604.

At 802, method 800 acquires patient scan data. The patient scan data may be raw scan data or a slice of a volume or image reconstruction. The patient scan data may be acquired by scanning a patient, or by accessing electronically stored data from a prior patient scan.

At 804, method 800 obtains unstandardized radiomics for the patient scan data acquired at 802. The unstandardized radiomics may be obtained by applying any radiomics technique to all or part of the patient scan data. By way of non-limiting example, the radiomics may be based on stochastic measures of image content, e.g., those derived from histograms and GLCMs.

At 806, method 800 determines standardized radiomics for the patient scan data. The standardized radiomics may be standardized in the sense that they may be effectively unaffected by the particular imaging machine used to produce them. The standardized radiomics may be determined using one or more of radiomics recovery model 204, 404, or 604. The standardized radiomics may thus be based on at least the patient scan data (e.g., \hat{μ} or y) and the unstandardized radiomics for the patient scan data (e.g., R(T,S)). According to embodiments that utilize radiomics recovery models 404 or 604, the standardized radiomics may be further based on analytical models of the imaging system and/or calibration phantom data for the particular imaging machine obtained using at least one calibration phantom, e.g., as shown and described in reference to FIGS. 5 and 7. Such calibration data may include noise S and resolution T for the particular imaging machine.

At 808, method 800 outputs the standardized radiomics. Method 800 may output the standardized radiomics by displaying on a computer monitor or other display device. Alternately, or in addition, method 800 may output the standardized radiomics by delivering them over a computer network, e.g., via email. Alternatively, or in addition, method 800 may output the standardized radiomics to radiomics models for clinical decision making.

FIG. 9 is a schematic diagram for a system 900 for implementing a method of radiomics standardization according to various embodiments. For example, system 900 may implement method 800 as shown and described above in reference to FIG. 8. System 900 includes imaging machine 902. Imaging machine 902 may be a computer tomography scanner, which includes a computer tomography gantry. The computer tomography scanner may by any of a variety of computer tomography scanners, including without limitation axial, helical, and cone-beam. Alternately, the imaging machine 902 may be an ultrasound machine, an MRI machine, a microscopy system, an x-ray machine, or an optical imaging system. The imaging machine 902 is communicatively coupled to computer system 906, either directly or via network 904, as shown. Computer system 906 includes input interface 908 at which patient scan data is received. Input interface 908 is communicatively coupled to one or more processors 910.

Processors 910 are communicatively coupled to random access memory 914 operating under control of or in conjunction with an operating system. The processors 910 in embodiments may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors 910 may communicate with data store 912, such as a hard drive or drive array, to access or store program instructions and other data. Processors 910 may, in general, be programmed or configured to execute control logic and control operations to implement methods disclosed herein, e.g., method 800 of FIG. 8. Other configurations of computer system 900, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A method of radiomics standardization for patient scan data obtained by a particular imaging device, the method comprising:
acquiring, using the particular imaging machine, the patient scan data;
obtaining unstandardized radiomics for the patient scan data;
recovering standardized radiomics for the patient scan data based on at least:
the patient scan data,
the unstandardized radiomics for the patient scan data, and
calibration phantom data for the particular machine obtained using at least one calibration phantom; and
outputting the standardized radiomics.

2. The method of claim 1, wherein the particular imaging machine comprises at least one: x-ray machine, computed tomography machine, magnetic resonance imaging machine, or ultrasound machine.

3. The method of claim 1, wherein the patient scan data comprises a two-dimensional slice of a three-dimensional volume constructed from raw patient scan data.

4. The method of claim 1, wherein the patient scan data comprises raw patient scan data.

5. The method of claim 1, further comprising:
providing the patient scan data and the calibration phantom data to a trained image property predictor; and
obtaining noise and resolution characteristics for the particular machine from the trained image property predictor;
wherein the recovering the standardized radiomics comprises recovering the standardized radiomics based on the patient scan data, the unstandardized radiomics for the patient scan data, and the noise and resolution characteristics.

6. The method of claim 1, wherein the recovering the standardized radiomics comprises:
providing the patient scan data, the unstandardized radiomics for the patient scan data, and the calibration phantom data for the particular machine to a machine learning model trained using a training corpus comprising radiomics in association with example scan data and calibration phantom data, whereby the machine learning model provides the standardized radiomics.

7. The method of claim 1, wherein the recovering comprises:
deblurring an image corresponding to the patient scan data to produce a deblurred image;
determining radiomics for the deblurred image;
determining radiomics for noise of the deblurred image; and
deconvolving the radiomics for the deblurred image with the radiomics for the noise of the deblurred image.

8. The method of claim 1, wherein the recovering comprises:
passing an image corresponding to the patient scan data to a first machine learning model trained to deblur images to obtain a deblurred image;
computing radiomics for the deblurred image;
passing the radiomics for the deblurred image to a second machine learning model trained to remove noise, whereby the standardized radiomics are obtained.

9. The method of claim 1, wherein the radiomics comprise standardized radiomics comprise a grey-level co-occurrence matrix.

10. The method of claim 1, wherein the outputting comprises causing the standardized radiomics to be input to a radiomics model for clinical decision making.

11. A system for radiomics standardization for patient scan data obtained by a particular imaging device, the system comprising at least one electronic processor that executes instructions to perform operations comprising:
acquiring the patient scan data produced by the particular imaging machine;
obtaining unstandardized radiomics for the patient scan data;
recovering standardized radiomics for the patient scan data based on at least:
the patient scan data,
the unstandardized radiomics for the patient scan data, and
calibration phantom data for the particular machine obtained using at least one calibration phantom; and
outputting the standardized radiomics.

12. The system of claim 11, wherein the particular imaging machine comprises at least one: x-ray machine, computed tomography machine, magnetic resonance imaging machine, or ultrasound machine.

13. The system of claim 11, wherein the patient scan data comprises a two-dimensional slice of a three-dimensional volume constructed from raw patient scan data.

14. The system of claim 11, wherein the patient scan data comprises raw patient scan data.

15. The system of claim 11, wherein the operations further comprise:
providing the patient scan data and the calibration phantom data to a trained image property predictor; and
obtaining noise and resolution characteristics for the particular machine from the trained image property predictor;
wherein the recovering the standardized radiomics comprises recovering the standardized radiomics based on the patient scan data, the unstandardized radiomics for the patient scan data, and the noise and resolution characteristics.

16. The system of claim 11, wherein the recovering the standardized radiomics comprises:
providing the patient scan data, the unstandardized radiomics for the patient scan data, and the calibration phantom data for the particular machine to a machine learning model trained using a training corpus comprising radiomics in association with example scan data and calibration phantom data, whereby the machine learning model provides the standardized radiomics.

17. The system of claim 11, wherein the recovering comprises:
deblurring an image corresponding to the patient scan data to produce a deblurred image;
determining radiomics for the deblurred image;
determining radiomics for noise of the deblurred image; and
deconvolving the radiomics for the deblurred image with the radiomics for the noise of the deblurred image.

18. The system of claim 11, wherein the recovering comprises:
passing an image corresponding to the patient scan data to a first machine learning model trained to deblur images to obtain a deblurred image;
computing radiomics for the deblurred image;
passing the radiomics for the deblurred image to a second machine learning model trained to remove noise, whereby the standardized radiomics are obtained.

19. The system of claim 11, wherein the radiomics comprise standardized radiomics comprise a grey-level co-occurrence matrix.

20. The method of claim 1, wherein the outputting comprises causing the standardized radiomics to be to be input to a radiomics model for clinical decision making.
